(19) United States
(12) Patent Application Publication
(10) Pub. No.: US 2019/0374468 A1
(43) Pub. Date: Dec. 12, 2019

(54) Liposomal Formulations for Allosteric AKT Inhibitors

(71) Applicant: Merck Sharp & Dohme Corp., Rahway, NJ (US)

(72) Inventors: Michael Hughes Smith, Waltham, MA (US); Marian Gindy, North Wales, PA (US)

(73) Assignee: Merck Sharp & Dohme Corp., Rahway, NJ (US)

(21) Appl. No.: 16/548,181

(22) Filed: Aug. 22, 2019

Related U.S. Application Data
(63) Continuation of application No. 15/103,911, filed on Jun. 13, 2016.
(60) Provisional application No. 61/916,875, filed on Dec. 17, 2013.

(51) Int. Cl. A61K 9/127; A61K 31/4375; A61K 9/00

(52) U.S. Cl. CPC A61K 9/1277; A61K 31/4375; A61K 9/1273; A61K 9/127; A61K 9/0019

(57) ABSTRACT

Disclosed herein is a lipid nanoparticle (LNP) composition comprising (a) an Akt inhibitor; (b) DSPC; (c) cholesterol; and (d) PEG-DMG. Also disclosed herein is a method for preparing the lipid composition using a scalable tangential flow micro-mixing technology.

### BACKGROUND OF THE INVENTION

Chemotherapeutic compounds are often associated with poor site-specific targeting and/or severe toxic side effects. For example, known compositions for allosteric Akt inhibitors are often associated with dose limiting toxicities (DLT) that limit the potential of these compounds for treating various cancers. The most frequent DLTs observed include skin rash, nausea, pruritus, hyperglycemia and diarrhea.

Liposomes have been used as a drug carrier for intravenously administered compounds. However, the use of liposomes for site-specific targeting via the bloodstream has been severely restricted by the rapid clearance of liposomes by cells of the reticuloendothelial system.

Encapsulation of antineoplastic compounds within liposomes has been used (see U.S. Pat. No. 5,213,804, “Solid Tumor Treatment Method and Composition”). However, achieving high encapsulation of a drug compound within the liposome is dependent on the physicochemical properties of the active ingredient and the behavior of the compound within the aqueous interior of the liposome.

There remains a need for improved lipid compositions for allosteric Akt inhibitors to improve efficacy and/or reduce off-target effects.

### SUMMARY OF THE INVENTION

Disclosed herein is a novel lipid nanoparticle (LNP) composition comprising: (a) an Akt inhibitor; (b) cholesterol; (c) 1,2-distearoyl-sn-glycero-3-phosphocholine (DSPC); and (d) 2-dimyristoyl-sn-glycerol methoxypolyethylene Glycol (PEG-DMG).

Also disclosed herein is a process for making an LNP composition of an Akt inhibitor using a scalable tangential flow micro-mixing technology.

### BRIEF DESCRIPTION OF THE FIGURES

FIG. 1: Top-view schematic of a Multi-Inlet Vortex Mixer (MIVM) impinging jet mixing device used in one embodiment of the invention, consisting of three aqueous flow streams (A) and one organic flow stream (O).

FIG. 2: Separation of LNP from free Compound B.

FIG. 3: Example HPLC chromatogram for fractionated lipid nanoparticles loaded with Compound B—analysis with charged aerosol detection.

FIG. 4: Example Cryo-TEM images of sulfate-encapsulation liposomes before (a and c) and after (b and d) Compound B encapsulation.

FIG. 5: Comparison of separated nanoparticles before and after dialysis purification.

### DETAILED DESCRIPTION OF THE INVENTION

Using lipid components that favor nanoparticle retention in circulation after intravenous (IV) dosing, the compositions disclosed herein enable the administration of Akt inhibitors with improved pharmacokinetics and/or reduced off-target effects.

In one embodiment, an LNP composition comprises (a) an Akt inhibitor; (b) DSPC; (c) cholesterol; and (d) PEG-DMG; wherein the Akt inhibitor is a compound of Formula C, or a tautomer thereof, or a pharmaceutically acceptable salt of a compound of Formula C or its tautomer:

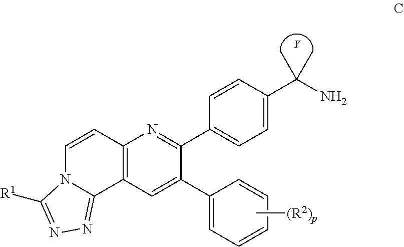

wherein: a is 0 or 1; b is 0 or 1; m is 0, 1 or 2; p is 0, 1 or 2;

R² is independently selected from: (C₁-C₆)alkyl, (C₁-C₆)alkoxy, —CO₂H, halo, —OH and —NH₂;

ring Y is (C₄-C₇)cycloalkyl;

R¹ is selected from: —H, oxo, (C═O)ₐO_{b}(C₁-C₁₀)alkyl, (C═O)ₐO_{b}-aryl, (C═O)ₐO_{b}(C₂-C₁₀)alkenyl, (C═O)ₐO_{b}(C₂-C₁₀)alkynyl, —CO₂H, halo, —OH, —O_{b}(C₁-C₆)perfluoroalkyl, (C═O)ₐNR⁷R⁸, CN, (C═O)ₐO_{b}(C₃-C₈)cycloalkyl, —S(O)ₘNR⁷R⁸, —SH, —S(O)ₘ—(C₁-C₁₀)alkyl and (C═O)ₐO_{b}-heterocyclyl, said alkyl, aryl, alkenyl, alkynyl, cycloalkyl, and heterocyclyl is optionally substituted with one or more substituents selected from R⁶;

R⁶ is: (C═O)ₐO_{b}C₁-C₁₀ alkyl, (C═O)ₐO_{b}aryl, C₂-C₁₀ alkenyl, C₂-C₁₀ alkynyl, (C═O)ₐO_{b} heterocyclyl, CO₂H, halo, —CN, —OH, —O_{b}C₁-C₆ perfluoroalkyl, —Oₐ(C═O)_{b}NR⁷R⁸, oxo, —CHO, —(N═O)R⁷R⁸, —S(O)ₘNR⁷R⁸, —SH, —S(O)ₘ—(C₁-C₁₀)alkyl or (C═O)ₐO_{b}C₃-C₈ cycloalkyl, said alkyl, aryl, alkenyl, alkynyl, heterocyclyl, and cycloalkyl optionally substituted with one or more substituents selected from R^{6a};

R^{6a} is selected from: (C═O)ₐO_{b}(C₁-C₁₀)alkyl, —Oₐ(C₁-C₃)perfluoroalkyl, (C₀-C₆)alkylene-S(O)ₘRₐ, —SH, oxo, —OH, halo, —CN, (C₂-C₁₀)alkenyl, (C₂-C₁₀)alkynyl, (C₃-C₆)cycloalkyl, (C₀-C₆)alkylene-aryl, (C₀-C₆)alkylene-heterocyclyl, (C₀-C₆)alkylene-N(R^{b})₂, C(O)Rₐ, (C₀-C₆)alkylene-CO₂Rₐ, C(O)H, and (C₀-C₆)alkylene-CO₂H, said alkyl, alkenyl, alkynyl, cycloalkyl, aryl, and heterocyclyl is optionally substituted with up to three substituents selected from R^{b}, OH, (C₁-C₆)alkoxy, halogen, CO₂H, CN, Oₐ(C═O)_{b}(C₁-C₆)alkyl, oxo, and N(R^{b})₂;

R⁷ and R⁸ are independently selected from: —H, (C═O)ₐO_{b}(C₁-C₁₀)alkyl, (C═O)ₐO_{b}(C₃-C₈)cycloalkyl, (C═O)ₐO_{b}-aryl, (C═O)ₐO_{b}-heterocyclyl, (C₂-C₁₀)alkenyl, (C₂-C₁₀)alkynyl, —SH, —SO₂Rₐ, and (C═O)ₐNR^{b}₂, said alkyl, cycloalkyl, aryl, heterocyclyl, alkenyl, and alkynyl is optionally substituted with one or more substituents selected from R^{6a}, or R⁷ and R⁸ can be taken together with the nitrogen to which they are attached to form a monocyclic or bicyclic heterocycle with 3-7 members in each ring and optionally containing, in addition to the nitrogen, one or two additional heteroatoms selected from N, O and S, said monocyclic or bicyclic heterocycle optionally substituted with one or more substituents selected from R^{6a};

R^{a} is (C₁-C₆)alkyl, (C₃-C₆)cycloalkyl, aryl, or heterocyclyl; and

R^{b} is independently: —H, (C₁-C₆)alkyl, aryl, heterocyclyl, (C₃-C₆)cycloalkyl, (C═O)ₐO_{b}(C₁-C₆)alkyl, or —S(O)ₘR^{a}.

When any variable (e.g. R²) occurs more than one time in any constituent, its definition on each occurrence is independent at every other occurrence. Also, combinations of substituents and variables are permissible only if such combinations result in stable compounds. Lines drawn into the ring systems from substituents represent that the indicated bond may be attached to any of the substitutable ring atoms. If the ring system is bicyclic, it is intended that the bond be attached to any of the suitable atoms on either ring of the bicyclic moiety.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be taken to be equivalent to the phrase “optionally substituted with at least one substituent” and in such cases the preferred embodiment will have from zero to four substituents, and the more preferred embodiment will have from zero to three substituents.

As used herein, “alkyl” is intended to include both branched and straight-chain saturated aliphatic hydrocarbon groups having the specified number of carbon atoms. For example, C₁-C₁₀, as in “(C₁-C₁₀)alkyl” is defined to include groups having 1, 2, 3, 4, 5, 6, 7, 8, 9 or 10 carbons in a linear or branched arrangement. For example, “(C₁-C₁₀)alkyl” specifically includes methyl, ethyl, n-propyl, i-propyl, n-butyl, t-butyl, i-butyl, pentyl, hexyl, heptyl, octyl, nonyl, decyl, and so on.

The term “cycloalkyl” means a monocyclic saturated aliphatic hydrocarbon group having the specified number of carbon atoms. For example, “cycloalkyl” includes cyclopropyl, methyl-cyclopropyl, 2,2-dimethyl-cyclobutyl, 2-ethyl-cyclopentyl, cyclohexyl, and so on.

“Alkoxy” represents either a cyclic or non-cyclic alkyl group of indicated number of carbon atoms attached through an oxygen bridge. “Alkoxy” therefore encompasses the definitions of alkyl and cycloalkyl above.

If no number of carbon atoms is specified, the term “alkenyl” refers to a non-aromatic hydrocarbon radical, straight, branched or cyclic, containing from 2 to 10 carbon atoms and at least one carbon to carbon double bond. Preferably one carbon to carbon double bond is present, and up to four non-aromatic carbon-carbon double bonds may be present. Thus, “(C₂-C₁₀)alkenyl” means an alkenyl radical having from 2 to 10 carbon atoms. Alkenyl groups include ethenyl, propenyl, butenyl, 2-methylbutenyl and cyclohexenyl. The straight, branched or cyclic portion of the alkenyl group may contain double bonds and may be substituted if a substituted alkenyl group is indicated.

The term “alkynyl” refers to a hydrocarbon radical straight, branched or cyclic, containing from 2 to 10 carbon atoms and at least one carbon to carbon triple bond. Up to three carbon-carbon triple bonds may be present. Thus, “(C₂-C₁₀)alkynyl” means an alkynyl radical having from 2 to 10 carbon atoms. Alkynyl groups include ethynyl, propynyl, butynyl, 3-methylbutynyl and so on. The straight, branched or cyclic portion of the alkynyl group may contain triple bonds and may be substituted if a substituted alkynyl group is indicated.

In certain instances, substituents may be defined with a range of carbons that includes zero, such as (C₀-C₆)alkylene-aryl. If aryl is taken to be phenyl (Ph), this definition would include phenyl itself as well as —CH₂Ph, —CH₂CH₂Ph, —CH(CH₃)CH₂CH(CH₃)Ph, and so on.

As used herein, “aryl” is intended to mean any stable monocyclic or bicyclic carbon ring of up to 7 atoms in each ring, wherein at least one ring is aromatic. Examples of such aryl elements include phenyl, naphthyl, tetrahydro-naphthyl, indanyl and biphenyl. In cases where the aryl substituent is bicyclic and one ring is non-aromatic, it is understood that attachment is via the aromatic ring.

The term heteroaryl, as used herein, represents a stable monocyclic or bicyclic ring of up to 7 atoms in each ring, wherein at least one ring is aromatic and contains from 1 to 4 heteroatoms selected from the group consisting of O, N and S. Heteroaryl groups within the scope of this definition include but are not limited to: acridinyl, carbazolyl, cinnolinyl, quinoxalinyl, pyrrazolyl, indolyl, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. As with the definition of heterocycle below, “heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring, respectively. Such heteroaryl moieties include but are not limited to: 2-benzimidazolyl, 2-quinolinyl, 3-quinolinyl, 4-quinolinyl, 1-isoquinolinyl, 3-isoquinolinyl and 4-isoquinolinyl.

The term “heterocycle” or “heterocyclyl” as used herein is intended to mean a 3- to 10-membered aromatic or nonaromatic heterocycle containing from 1 to 4 heteroatoms selected from the group consisting of O, N and S, and includes bicyclic groups. “Heterocyclyl” therefore includes the above mentioned heteroaryls, as well as dihydro and tetrahydro analogs thereof. Further examples of “heterocyclyl” include, but are not limited to the following: benzoimidazolyl, benzoimidazolonyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof. Attachment of a heterocyclyl substituent can occur via a carbon atom or via a heteroatom.

As appreciated by those of skill in the art, “halo” or “halogen” as used herein is intended to include chloro (Cl), fluoro (F), bromo (Br) and iodo (I).

The LNP compositions disclosed herein have unexpected physical and chemical properties that enable IV delivery of the Akt inhibitors with improved efficacy and/or reduced off-target effects.

In one embodiment, the Akt inhibitor is a compound of Formula D, or a tautomer thereof, or a pharmaceutically acceptable salt of a compound of Formula D or its tautomer:

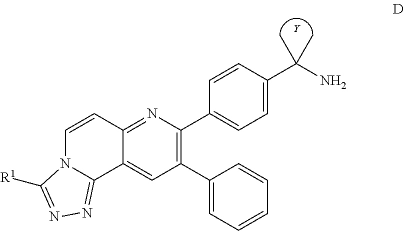

wherein ring Y is cyclobutyl; and

R¹ is —H, pyrimidyl, —OH, methyl or cyclopropyl.

In one embodiment, the Akt inhibitor is 8-[4-(1-aminocyclobutyl)phenyl]-9-phenyl[1,2,4]triazolo[3,4-f]-1,6-naphthyridin-3-ol having Formula A below (hereinafter, “Compound A”), or a pharmaceutically acceptable salt thereof:

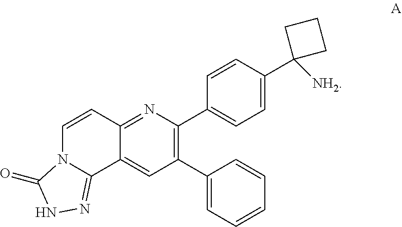 [SMILES: NC1(c2ccc(-c3nc4ccn5c(=O)[nH]nc5c4cc3-c3ccccc3)cc2)CCC1]

The pharmaceutically acceptable salts of the instant compounds can be synthesized from the compounds disclosed herein which contain a basic moiety by conventional chemical methods. Generally, the salts of the basic compounds are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base.

Thus, pharmaceutically acceptable salts of the compounds of this invention include the conventional non-toxic salts of the compounds disclosed herein as formed by reacting a basic instant compound with an inorganic or organic acid. For example, conventional non-toxic salts include those derived from inorganic acids such as hydrochloric, hydrobromic, sulfuric, sulfamic, phosphoric, and nitric acid; as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, and trifluoroacetic (TFA).

In one embodiment, the Akt inhibitor is the hydrochloride salt of Compound A having Formula B (hereinafter, “Compound B”):

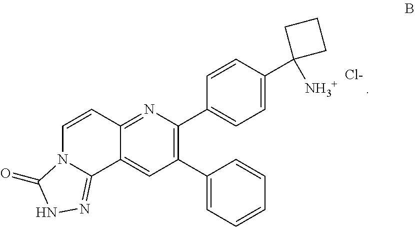 [SMILES: [NH3+]C1(c2ccc(-c3nc4ccn5c(=O)[nH]nc5c4cc3-c3ccccc3)cc2)CCC1 ; [Cl-]]

Compounds illustrated by Formulae A, B, C and D are allosteric Akt inhibitors with nanomolar potency against human Akt1, an anti-apoptotic factor. Without wishing to be bound by theory, it is believed that Akt1 has a central role in the cellular response to oxidative and osmotic stress, irradiation, chemotherapy, and ischemic shock. Overexpression of that factor is associated with multiple cancer phenotypes and frequently results in drug resistance. When administered alone, certain compounds as illustrated by Formula A, B, C and D have shown antitumor activity both in vitro and in vivo, while also demonstrating synergistic effects with conventional antineoplastic compounds and targeted therapies. Compounds of Formulae A, B, C and D can be prepared using synthetic procedures and conditions as described in U.S. patent application Ser. No. 11/999,234, the entire content of which is incorporated herein.

Using the instantly disclosed LNP compositions, site specific delivery of the Akt inhibitors to solid tumors reduces off-target effects and toxicity. In a series of proof-of-concept syntheses, remote loading was used as an effective means to encapsulate an Akt inhibitor with high total encapsulation of the compound and pH-controlled release.

In one embodiment, the chemical structures of the components of an LNP composition disclosed herein are shown in Table 1.

TABLE 1
Chemical Structures of the Components of an LNP composition

| Component | Chemical Structure |
|---|---|
| Compound B | 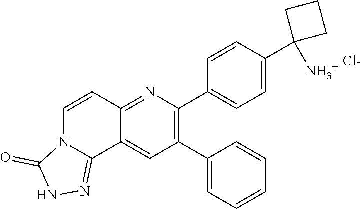 [SMILES: [NH3+]C1(c2ccc(-c3nc4ccn5c(=O)[nH]nc5c4cc3-c3ccccc3)cc2)CCC1 ; [Cl-]] |
| Cholesterol | 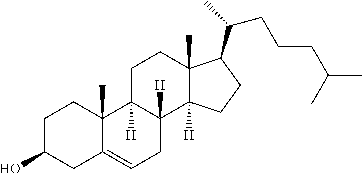 |
| DSPC | 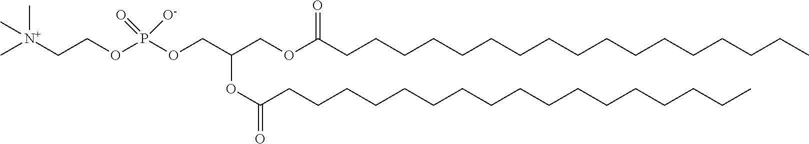 [SMILES: CCCCCCCCCCCCCCCCCC(=O)OCC(COP(=O)([O-])OCC[N+](C)(C)C)OC(=O)CCCCCCCCCCCCCCCCC] |
| PEG-DMG | 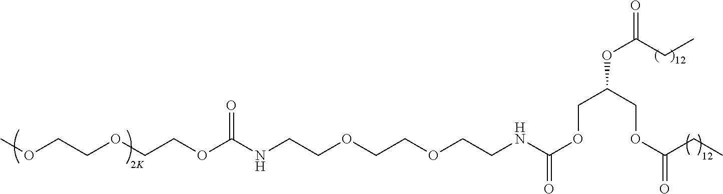 |

In one embodiment, the molar ratio of the lipid components DSPC:cholesterol:PEG-DMG in the above LNP compositions is 55-70:20-40:1-15.

In one embodiment, the molar ratio of the lipid components DSPC:cholesterol:PEG-DMG in the above LNP compositions is 60-70:25-35:2-10.

In one embodiment, the molar ratio of the lipid components DSPC: cholesterol:PEG-DMG in the above LNP compositions is 60-65:25-30:5-10.

In one embodiment, the molar ratio of the lipid components DSPC:cholesterol:PEG-DMG in the above LNP compositions is 65:30:5.

In one embodiment, the molar ratio of the lipid components DSPC:cholesterol:PEG-DMG in the above LNP compositions is 60:38:2.

In one embodiment, the molar ratio of the lipid components DSPC:cholesterol:PEG-DMG in the above LNP compositions is 60:35:5.

In one embodiment, the molar ratio of the lipid components DSPC:cholesterol:PEG-DMG in the above LNP compositions is 65:33:2.

In one embodiment, the Akt inhibitor is present at 0.1-10 mg/mL of the final composition.

In one embodiment, the Akt inhibitor is present at 1-5 mg/mL of the final composition.

In one embodiment, the Akt inhibitor is present at 1-3 mg/mL of the final composition.

In one embodiment, the Akt inhibitor is present at 1 mg/mL of the final composition.

In one embodiment, the LNP composition is used for intravenous administration.

In one embodiment, a pharmaceutical formulation comprises the LNP composition described above and further comprises a cryoprotectant selected from sucrose, trehalose, raffinose, stachyose, verbascose, mannitol, glucose, lactose, maltose, maltotriose-heptaose, dextran, hydroxyethyl starch, insulin, sorbitol, glycerol, arginine, histidine, lysine, proline, dimethylsulfoxide and any combination thereof.

In one embodiment, the cryoprotectant is sucrose.

In one embodiment, the cryoprotectant is trehalose.

In one embodiment, the cryoprotectant is a combination of sucrose and trehalose.

The LNP compositions disclosed herein can be prepared using a scalable and robust nanoprecipitation method in a micro-mixing device. In one embodiment, using a Multi-Inlet Vortex Mixer (MIVM), nanometric particles of organic compounds with mixed solubility can be formed, including polymers and lipids. Importantly, the method is rapid and allows controlled mixing at high solute concentrations.

As used herein, a “mixer” refers to a device with three or more inlets meeting in a central mixing chamber designed to enhance mixing, and a single outlet. In one embodiment, a “mixer” refers to a MIVM device with four inlets, meeting in a central mixing chamber, and a single outlet. For a detailed description of the MIVM method and device, see U.S. patent application Ser. No. 13/639,628, the entire content of which is incorporated herein.

In one embodiment, a method for preparing an LNP composition of the invention comprises:

a) providing one or more aqueous solutions in one or more reservoirs, one solution per reservoir;

b) providing one or more organic solutions in one or more reservoirs, one solution per reservoir, wherein one or more of the organic solutions comprise a lipid;

c) mixing the one or more aqueous solutions with the one or more organic solutions in a first mixing region, wherein the first mixing region is a MIVM; and

wherein the one or more aqueous solutions and the one or more organic solutions are introduced tangentially into a mixing chamber within the MIVM so as to substantially instantaneously produce a lipid nanoparticle solution; and

d) dissolving an Akt inhibitor in the lipid nanoparticle solution to form an LNP composition encapsulating the Akt inhibitor.

In one embodiment of the above method, the one or more aqueous solutions and the one or more organic solutions in step c) are introduced tangentially into a mixing chamber within the MIVM so as to substantially instantaneously produce the lipid nanoparticle solution.

In one embodiment of the above method, one or more of the aqueous solutions of step a) comprise 10 to 500 mM ammonium sulfate. In one embodiment, one or more of the aqueous solutions comprise 50 to 300 mM ammonium sulfate. In one embodiment, one or more of the aqueous solutions comprise 50 to 200 mM ammonium sulfate. In one embodiment, one or more of the aqueous solutions comprise 50 to 150 mM ammonium sulfate.

In one embodiment, one or more of the aqueous solutions comprise 150 mM ammonium sulfate. Sucrose can be added to maintain comparable internal osmotic pressure across the compositions.

In one embodiment of the above method, the lipid nanoparticle solution of step c) is purified by dialysis or tangential flow filtration into either pH 3.0 citrate buffer or pH 3.0 sucrose to remove free ammonium sulfate before being used in step d).

In one embodiment, the lipid nanoparticle solution of step c) is purified by dialysis into a pH 3.0 citrate buffer to remove free ammonium sulfate before being used in step d).

In one embodiment, the lipid nanoparticle solution of step c) is purified by dialysis into pH 3.0 sucrose to remove free ammonium sulfate before being used in step d).

In one embodiment, the lipid nanoparticle solution of step c) is purified by tangential flow filtration into a pH 3.0 citrate buffer to remove free ammonium sulfate before being used in step d).

In one embodiment, the lipid nanoparticle solution of step c) is purified by tangential flow filtration into pH 3.0 sucrose to remove free ammonium sulfate before being used in step d).

In one embodiment, the Akt inhibitor in step (d) is present at a concentration of 0.1 to 15 mg/mL in the final composition. In another embodiment, the Akt inhibitor in step (d) is present at a concentration of 0.1 to 10 mg/mL in the final composition. In another embodiment, the Akt inhibitor in step (d) is present at a concentration of 0.5 to 5 mg/mL in the final composition.

In one embodiment of the above method, the Akt inhibitor is Compound A or Compound B. In one embodiment, Compound A or Compound B is present at a concentration of 0.1 to 10 mg/mL in the final composition.

In one embodiment of the above method, the Akt inhibitor is Compound B present at a concentration of 0.5 to 5 mg/mL in the final composition.

In one embodiment of the method, the Akt inhibitor is Compound B present at a concentration of 0.5 to 3 mg/mL in the final composition.

In one embodiment of the method, the Akt inhibitor is Compound B present at a concentration of 1 to 3 mg/mL in the final composition.

In one embodiment of the method, the Akt inhibitor is Compound B present at a concentration of 1 mg/mL in the final composition.

In one embodiment of the method, the Akt inhibitor is Compound B present at a concentration of 0.5 mg/mL in the final composition.

In one embodiment of the method, the Akt inhibitor is Compound B present at a concentration of 0.1 mg/mL in the final composition.

In one embodiment of the above method, the solution of step d) containing the Akt inhibitor is heated at 30-70° C. In one embodiment, the solution is heated with stirring.

In one embodiment of the method, the solution of step d) containing the Akt inhibitor is heated at 50-70° C. In one embodiment, the solution is heated with stirring.

In one embodiment of the method, the solution of step d) containing the Akt inhibitor is heated at 55-66° C. In one embodiment, the solution is heated with stirring.

In one embodiment of the method, the solution of step d) containing the Akt inhibitor is heated at 60° C. In one embodiment, the solution is heated with stirring.

In one embodiment of the above method, the solution of step d) containing the Akt inhibitor is heated for 0.25-5 hours. In one embodiment, the solution is heated with stirring.

In one embodiment, the solution of step d) containing the Akt inhibitor is heated for 0.5-4 hours. In one embodiment, the solution is heated with stirring.

In one embodiment, the solution of step d) containing the Akt inhibitor is heated for 2-4 hours. In one embodiment, the solution is heated with stirring.

In one embodiment, the solution of step d) containing the Akt inhibitor is heated for 3 hours. In one embodiment, the solution is heated with stirring.

In one embodiment of the above method, the solution of step d) containing the Akt inhibitor is heated at 50-65° C. for 1-4 hours. In one embodiment, the solution is heated with stirring.

In one embodiment, the solution of step d) containing the Akt inhibitor is heated at 55-65° C. for 2-4 hours. In one embodiment, the solution is heated with stirring.

In one embodiment, the solution of step d) containing the Akt inhibitor is heated at 60° C. for 2-4 hours. In one embodiment, the solution is heated with stirring.

In one embodiment, the solution of step d) containing the Akt inhibitor is heated at 60° C. for 3 hours. In one embodiment, the solution is heated with stirring.

In one embodiment of the above method, the heated solution from the previous step is cooled to about 5-30° C. after heating.

In one embodiment of the method, the heated solution is cooled to about 20-25° C. after heating.

In one embodiment of the method, the heated solution is cooled to about 22° C. after heating.

In one embodiment, a method for preparing an LNP composition of the invention comprises:

a) providing three aqueous solutions in one or more reservoirs, wherein one or more of the aqueous solutions comprise 50 to 300 mM ammonium sulfate;

b) providing one organic solution in a reservoir, wherein the organic solution comprises DSPC, cholesterol and PEG-DMG;

c) mixing the three aqueous solutions with the organic solution in a first mixing region, wherein the first mixing region is a Multi-Inlet Vortex Mixer (MIVM); and

wherein the three aqueous solutions and the one organic solution are introduced tangentially into a mixing chamber within the MIVM so as to substantially instantaneously produce a lipid nanoparticle solution;

d) purifying the lipid nanoparticle solution from the previous step to remove free ammonium sulfate; and

e) dissolving Compound A or Compound B in the purified lipid nanoparticle solution to form an LNP composition encapsulating Compound A or Compound B.

In one embodiment of the above method, the three aqueous solutions and the one organic solution in step c) are introduced tangentially into a mixing chamber within the MIVM so as to substantially instantaneously produce a lipid nanoparticle solution.

In one embodiment of the above method, one or more of the aqueous solutions of step a) comprise 50 to 300 mM ammonium sulfate. In another embodiment, one or more of the aqueous solutions of step a) comprise 150 mM ammonium sulfate. In another embodiment, one or more of the aqueous solutions of step a) comprise sucrose. In another embodiment, one or more of the aqueous solutions of step a) comprise ammonium sulfate and sucrose. Sucrose was added to maintain comparable internal osmotic pressure across the compositions.

In one embodiment of the above method, the lipid nanoparticle solution of step c) is purified by dialysis or tangential flow filtration into either pH 3.0 citrate buffer or pH 3.0 sucrose to remove free ammonium sulfate in step d).

In one embodiment, the lipid nanoparticle solution of step c) is purified by dialysis into a pH 3.0 citrate buffer to remove free ammonium sulfate in step d).

In one embodiment, the lipid nanoparticle solution of step c) is purified by dialysis into pH 3.0 sucrose to remove free ammonium sulfate in step d).

In one embodiment, the lipid nanoparticle solution of step c) is purified by tangential flow filtration into a pH 3.0 citrate buffer to remove free ammonium sulfate in step d).

In one embodiment, the lipid nanoparticle solution of step c) is purified by tangential flow filtration into pH 3.0 sucrose to remove free ammonium sulfate in step d).

In one embodiment of the method, the Akt inhibitor is Compound A or Compound B. In one embodiment, Compound A or Compound B in step (e) is present at a concentration of 0.1 to 10 mg/mL in the final composition. In another embodiment, Compound A or Compound B in step (e) is present at a concentration of 0.1 to 5 mg/mL in the final composition. In another embodiment, Compound A or Compound B in step (e) is present at a concentration of 0.5 to 5 mg/mL in the final composition.

In one embodiment of the method, the Akt inhibitor is Compound A or Compound B present at a concentration of 0.5 to 3 mg/mL in the final composition.

In one embodiment of the method, the Akt inhibitor is Compound A or Compound B present at a concentration of 1 to 3 mg/mL in the final composition.

In one embodiment of the method, the Akt inhibitor is Compound A or Compound B present at a concentration of 1 mg/mL in the final composition.

In one embodiment of the method, the Akt inhibitor is Compound A or Compound B present at a concentration of 0.5 mg/mL in the final composition.

In one embodiment of the method, the Akt inhibitor is Compound A or Compound B present at a concentration of 0.1 mg/mL in the final composition.

In one embodiment of the above method, the solution of step d) containing Compound A or Compound B is heated at 50-70° C. In one embodiment, the solution is heated with stirring.

In one embodiment of the method, the solution of step d) containing Compound A or Compound B is heated at 55-65° C. In one embodiment, the solution is heated with stirring.

In one embodiment of the method, the solution of step d) containing Compound A or Compound B is heated at 60° C. In one embodiment, the solution is heated with stirring.

In one embodiment of the above method, the solution of step d) containing Compound A or Compound B is heated at 55-65° C. for 1-4 hours. In one embodiment, the solution is heated with stirring.

In one embodiment, the solution of step d) containing Compound A or Compound B is heated at 55-60° C. for 2-4 hours. In one embodiment, the solution is heated with stirring.

In one embodiment, the solution of step d) containing Compound A or Compound B is heated at 60° C. for 3 hours. In one embodiment, the solution is heated with stirring.

In one embodiment of the above method, the heated solution from the previous step is cooled to about 20-25° C. after heating.

In one embodiment, the heated solution is cooled to about 22° C. after heating.

One embodiment of the invention includes methods for treating a human or animal cancer comprises intravenously administering the lipid composition disclosed herein to said human or animal.

One embodiment of the inventions includes uses of the lipid composition disclosed herein for the manufacture of a medicament for treating a human or animal cancer comprising intravenous administration of said composition to said human or animal.

### EXAMPLES

Examples provided are intended to assist in a further understanding of the invention. Particular materials used, species and conditions are intended to be further illustrative of the invention and not limitative of the reasonable scope thereof. The lipid components used herein are either commercially available or are readily prepared by one of ordinary skill in the art.

### Lipid Composition Preparation

The following assay/reagents were used in the following examples and/or procedures:

Lipid Components

| | |
|---|---|
| Cholesterol: | 30 mole % |
| PEG-DMG | 5 mole % |
| DSPC | 65 mole % |

Aqueous Components (Internal Liposomal Compartment)

pH 3.0 citrate buffer 20 mM

10-150 mM sucrose

50-150 mM ammonium sulfate

Loading Conditions and Active Agent (External to Liposomes in Composition)

pH 3.0 citrate buffer 20 mM

1-3 mg/mL of Compound B

As a general procedure for preparing an LNP composition, lipid components were dissolved in ethanol (EtOH) and aqueous components were dissolved in distilled, deionized water. The aqueous stream may contain variable concentrations of ammonium sulfate ((NH₄)₂SO₄) to adjust total loading of Compound B. In one embodiment, 150 mM of (NH₄)₂SO₄ is used. Sucrose may be added to balance osmotic pressure in some examples.

For mixing, the solution composition contained 25% EtOH/lipid mixture (1 impinging stream) and 75% aqueous feed (split among 3 impinging streams, see FIG. 1). Solutions were mixed in the tangential flow mixer chamber of MIVM at a total flow rate of 240 mL/min. Total flow rate may be adjusted to modulate particle size and polydispersity index. Streams may be modified to include additional stabilizers or excipients.

An additional aqueous solution (pH 3.0 citrate) was administered downstream to dilute the organic solvent component (EtOH) to a final concentration of 12.5%. This dilution step may enhance colloidal stability by reducing kinetics of Ostwald ripening in some compositions.

Following synthesis, liposomes containing ammonium sulfate were purified by dialysis or tangential flow filtration into either pH 3.0 citrate buffer or pH 3.0 sucrose (292 mM) to remove free sulfate.

Loading was achieved by first dissolving Compound B in the purified liposome component at a concentration of 1-5 mg/mL. The solution was rapidly stirred at 60° C. for 1 hour (300 RPM) and subsequently cooled to 22° C. After loading, the particles were analyzed for Compound B encapsulation. Particles were further purified after loading via dialysis or tangential flow filtration.

In the following section, two different methods for the synthesis of precursor liposomes suitable for encapsulation of Compound B are described—a conventional thin film hydration/extrusion method and a novel MIVM method utilizing flash nanoprecipitation.

LNP Preparation by Thin Film Hydration/Extrusion—

Lipid components were dissolved in chloroform at a molar ratio of 65:30:5 (DSPC:Chol:PEG-DMG) to a total lipid concentration of 2 mM. Solvent evaporation was performed by rotovap, followed by rehydration in an aqueous mixture of 150 mM sucrose and 150 mM (NH₄)₂SO₄ (adjusted to pH 3.0 with citrate 30 mM). Nanometer-scale vesicles were prepared by high pressure extrusion (5 passes, 1,000 atm) through 0.1 μm filters. To remove free sulfate, lipid nanoparticles were dialyzed (100 kDa MWCO membrane) against 300 mM sucrose solution (pH 5.5, un-buffered).

LNP Preparation by Thin Flash Nanoprecipitation Using MIVM—

A scaled lipid nanoparticle preparation method was achieved via Multi-Inlet Vortex Mixer (MIVM), enabling the production of lipid nanoparticles at high concentration with uniform size. Four-stream MIVM configuration as depicted in FIG. 1 was used.

The organic phase flow stream (0) was prepared by dissolving the lipid components in anhydrous ethanol to a total lipid concentration of 22.3 mM (Table 2). The aqueous phase (A) was prepared by dissolving (NH₄)₂SO₄ in pH-corrected distilled, deionized water to a concentration of 150 mM (pH=3.0) and subsequently filtered through a 0.8/0.2 Acrodisc syringe filter.

TABLE 2
Lipid composition and material information

| Component | Batch | Mol % | MW |
|---|---|---|---|
| Cholesterol | 043 | 30 | 387 |
| PEG-DMG | 003 | 5 | 2837 |
| DSPC | 014 | 65 | 790 |

The organic and aqueous flow streams were mixed at a controlled rate utilizing the MIVM device and a PHD 2000 Ultra syringe pump. In one embodiment, flow streams were mixed at a flow rate of 60 mL/min per syringe (total flow rate=240 mL/min), collecting 80 mL of total product volume. Lipid nanoparticles were filtered through a 0.8/0.2 Acrodisc filter prior to purification via tangential flow filtration (membrane MWCO=100 kDa). In those purification steps, excess sulfate and ethanol were removed by multiple buffer replacements with 292 mM sucrose, pH 3.0.

Encapsulation of Compound B into Prepared Liposomes

In a typical loading procedure, dry powder of Compound B was dissolved to a concentration of 2.0 mg/mL in the sulfate-containing liposome composition. While mixing in a round-bottom glass, the suspension was heated to 60° C. for 3 hours in a temperature-controlled water bath and subsequently cooled to 22° C. Loaded particles were filtered through 0.2 μm Acrodisc filters prior to analytical analysis.

This LNP composition has the following advantages: 1) increased Compound B solubility during loading steps (low ionic and buffer strength); and 2) sustained physical stability of liposomes through osmotic pressure balancing via sucrose solute content. Particles were concentrated via tangential flow filtration to a total lipid concentration of 22 mg/mL and were filtered (0.2 μm Acrodisc) prior to storage at 4° C.

### Particle Characterization

Particle diameter and size distribution were measured via dynamic light scattering using the DynaPro Plate Reader (Wyatt Technology, Santa Barbara, Calif.). Two-dimensional chromatography was performed for each sample using a Dionex Ultimate 3000 2D UHPLC (Dionex, Sunnyville, Calif.). The first dimension utilized Size Exclusion Chromatography (SEC) to separate the lipid nanoparticles from any free Compound B in the sample. An isocratic flow rate at 1.0 mL/min of PBS pH 7.5 was used with a Zorbax GF-450, 6 μm, 9.4×250 mm column (Agilent Technologies, Santa Clara, Calif.) at ambient temperature. This first dimension was fractionated based on change of slope in the chromatogram triggering a second dimension that was automatically injected onto a RRHD SB-C18 reverse phase column (Agilent Technologies, Santa Clara, Calif.). A gradient of 0.1% TFA in water and 0.1% TFA in methanol at 1.0 mL/min was implemented for the reverse phase dimension at 80° C., and detected using a charged aerosol detector (CAD) (Dionex, Sunnyville, Calif.).

### Particle Imaging by Crvo-TEM

Undiluted samples of lipid nanoparticles were suspended on a Quantifoil holey carbon film supported by a 300 mesh copper grid (Quantifoil Micro Tools GmbH, Germany) previously cleaned by a glow discharge plasma treatment. Samples were vitrified using an FEI Vitrobot Mark IV operating at 4° C. and 95% relative humidity. Vitrified samples were stored and transferred under a liquid nitrogen atmosphere in order to maintain the temperature at or below −165° C. at all times. Imaging was performed on an FEI Tecnai Spirit Biotwin transmission electron microscope operating at 100 keV and equipped with a Gatan Ultrascan 1000 2MP under mounted CCD camera. The distribution of particles and overall sample quality were observed at 4,800× magnification (2.1 nm/pixel) and particle morphology and structure were observed at 30,000× (0.35 nm/pixel) magnification. Images were acquired with electron doses between 6.5 and 10 e⁻/Å².

### Results

### Solubility of Compound B

The solubility of Compound B showed pH-dependence, with highest solubilities observed in acidic environments (Table 3).

TABLE 3
Aqueous solubility of Compound B

| pH | Buffer | Solubility |
|---|---|---|
| 2.1 | 0.01N HCl | 4.23 |
| 2.8 | 0.1M Citrate | 10.6 |
| 3.9 | 0.1M Citrate | 0.17 |
| 5.0 | 0.1M Citrate | 0.059 |
| 5.6 | 0.1M Phosphate | 0.14 |
| 6.9 | 0.1M Phosphate | 0.036 |
| 8.6 | 0.1M Carbonate | 0.0007 |
| 4.1 | Un-buffered | 7.46 |

In addition to pH-dependent solubility, Compound B was highly sensitive to the identity and concentration of buffering salts. As depicted in Table 3, high solubilities were achieved in un-buffered media (i.e. distilled, deionized water), despite a pH value of 4.

The behavior of Compound B in the presence of sulfate salts was further evaluated visually. Vials containing 1 mg/mL Compound B in pH 3.0 citrate buffer with 0.1 M, 0.25 M, 0.5 M, and 1.0 M, respectively, of (NH₄)₂SO₄ were visually observed at 20° C. Compound B dissolved in acidic aqueous buffers had a clear, strong yellow appearance. The addition of ammonium sulfate resulted in rapid flocculation (<30 min) for Compound B solutions and a distinct change in appearance. Solutions became increasingly more viscous with a white/opaque appearance with increasing amounts of ammonium sulfate.

From the outcomes shown in Table 3 and the visual observations, it is hypothesized that salt-dependent precipitation behaviors may enable encapsulation via remote loading for Compound B.

### LNP Composition Prepared Using a Conventional Liposome Preparation Method

Lipid nanoparticles were prepared by thin film hydration and repeated extrusion, as described above. Free ammonium sulfate was removed by extensive dialysis into isotonic sucrose solution (300 mM, pH 3.0 un-buffered). The hydrodynamic diameter (Dₕ) of resulting particles were larger than the extruded pore size (˜100 nm), presumably due to the high osmotic pressure inside the liposome resulting from encapsulated sucrose and ammonium sulfate (Table 4, control). The particles were loaded by the addition of Compound B and heating to 60° C. for 3 hr. After heating and returning the solutions to ambient laboratory temperature (22° C.), a noticeable change in sample turbidity was observed.

TABLE 4
Particle characterization via dynamic light scattering for loaded particles.

| [Compound B]_{loading} | Loading Temp | Dₕ | PD |
|---|---|---|---|
| (mM) | (° C.) | (nm) | (nm) |
| Control (no Compound B) | 60 | 175 ± 2 | 15 ± 9 |
| 2.3 | 60 | 157 ± 4 | 27 ± 9 |
| 4.5 | 60 | 160 ± 2 | 28 ± 5 |
| 6.8 | 60 | 163 ± 2 | 23 ± 7 |
| 6.8 | 22 | 173 ± 1 | 17 ± 5 |

The increase in turbidity observed from incubation with Compound B was indicative of an increase in scattering from the colloidal dispersion, either due to an increase in particle molar mass, particle size, or aggregation/flocculation of the colloid in solution. It is worth noting that despite the increased turbidity, the Dₕ of particles remained comparable to particles without Compound B. However, a notable increase in size-dispersity (PD) was observed. To quantify encapsulation, liposomes were first separated from free Compound B via size exclusion chromatography (SEC) with UV detection.

In comparison to particles loaded at room temperature, incubation at 60° C. during the loading step resulted in a decrease in the free Compound B peak (retention time=˜32 min) (FIG. 2). The area reduction of free Compound B (19%) is likely a result of encapsulation within the liposomes. In contrast, a large increase in the peak area associated with the liposome (˜276%) was observed (retention time=˜7.5 min). The increase in absorbance observed for the lipid nanoparticles also suggests sequestration of Compound B within the carrier vehicle.

Separated lipid particle fractions were diluted in a TFA/methanol gradient in a second dimension

Despite differences in total Compound B encapsulation, the total concentration and relative abundance of lipid components were similar for particles under the two loading temperatures. Additionally, no difference in lipid composition between loaded and non-loaded liposomes was detected, as shown in Table 5.

TABLE 5
Measured lipid composition for control and remote-loaded lipid
nanoparticles (Compound B RLL).

| | | PEG DMG | DSPC |
|---|---|---|---|
| | Chol (mg/mL) | (mg/mL) | (mg/mL) |
| Empty LNP | 3.94 | 4.13 | 16.25 |
| Compound B RLL | 3.95 | 4.16 | 16.11 |

FIG. 3 shows an example HPLC chromatogram for fractionated lipid nanoparticles loaded with Compound B at 22° C. (solid line) or 60° C. (dotted line) for 1 hour. Much higher concentrations of Compound B were observed for particles loaded at 60° C. Using a second dimension analysis for separated particles, the total concentration of Compound B present within the lipid fraction was 0.3 mg/mg lipid. With particle size, size-dispersity, and particle concentration as input variables, an internal Compound B concentration of 79 mg/mL was calculated, which far exceeds the solubility limit of Compound B in all tested aqueous media. This result suggested that high loading is probably driven by Compound B precipitation within the lipid nanoparticles.

The morphology of the empty and loaded lipid nanoparticles was qualitatively assessed by Cryo-TEM as shown in FIG. 4. Before encapsulation, the lipid nanoparticle population consisted of single and multi-lamellar vesicles, ranging in diameter from ˜120-200 nm. High contrast was observed within the interior of LNPs, likely due to the high concentration of sucrose and sulfate encapsulated within the particle interior. After loading, a mixture of spherical and ellipsoid vesicles was present across all microscopy images (b and d). The rod-like appearance of vesicles as a result of active loading has been attributed to the formation of crystalline Compound B within the particles, causing membrane elongation to accommodate the phase change of the drug.

### LNP Composition Prepared Using a Scalable Method (MIVM)

In an effort to scale the preparation of remote-loaded liposomes, sulfate-encapsulated liposomes were prepared by nanoprecipitation via Multi-Inlet Vortex Mixer (MIVM) (FIG. 1). Liposomes were formulated with identical lipid components as the particles prepared by conventional extrusion. However, sucrose concentration was reduced from the internal aqueous solution in order to reduce particle osmotic pressure and attain sizes amenable to tumor localization via EPR (˜100 nm).

TABLE 6
Particle characterization via DLS for liposomes assembled by
multi-inlet vortex mixer.

| | Mixer Flow Rate | [(NH₄)₂SO₄] | [sucrose] | Dₕ | PD |
|---|---|---|---|---|---|
| Sample | (mL/min) | (mM) | (mM)* | (nm) | (nm) |
| 1 | 120 | 150 | 0 | 111 | 25 |
| 2 | 180 | 150 | 0 | 108 | 24 |
| 3 | 240 | 150 | 0 | 95 | 20 |
| 4 | 240 | 100 | 150 | 100 | 23 |
| 5 | 240 | 50 | 400 | 103 | 24 |

*Sucrose was added to maintain comparable internal osmotic pressure across the compositions.

Lipid composition and total loading was comparable for samples prepared using the MIVM method of preparation. In one embodiment (Sample 1), lipid nanoparticles were separated from free Compound B via size-exclusion chromatography both before and after purification (FIG. 5).

As can be seen from FIG. 5, comparison separated nanoparticles before (solid line) and after (dotted line) dialysis purification reveals significant loss of free Compound B (peak 2) and retention of encapsulated Compound B (peak 1).

The total lipid composition was maintained utilizing the MIVM method for liposome synthesis (Table 7).

TABLE 7
Lipid composition of sulfate-loaded particles (150 mM) prepared via
multi-inlet vortex mixing at a total flow rate of 240 mL/min.

| | mol % | mol % | |
|---|---|---|---|
| | formulated | measured | concentration |
| Component | (%) | (%) | (mg/mL) |
| Cholesterol | 30 | 31.8 | 3.9 |
| PEG-DMG | 5 | 4.5 | 4.1 |
| DSPC | 65 | 63.6 | 16.3 |

This MIVM preparation method is amenable to industrial scale up and can produce high quality lipid compositions of Akt inhibitors.

One skilled in the art would readily appreciate that the present invention is well adapted to carry out the objects and obtain the ends and advantages mentioned, as well as those inherent therein. The methods and compositions described herein, as presently representative of preferred embodiments, are exemplary and are not intended as limitations on the scope of the invention. Changes therein and other uses will occur to those skilled in the art, which are encompassed within the spirit of the invention, are defined by the scope of the claims.

1-17. (canceled)

18. A lipid nanoparticle composition comprising:
(a) an active agent;
(b) 1,2-distearoyl-sn-glycero-3-phosphocholine (DSPC);
(c) cholesterol; and
(d) 1,2-dimyristoyl-sn-glycerol methoxypolyethylene Glycol (PEG-DMG);
wherein the molar ratio of the lipid components DSPC:cholesterol:PEG-DMG is 60-70:25-35:2-10.

19. The composition of claim 18, wherein the active agent of (a) is an AKT inhibitor.

20. The composition of claim 18, wherein the molar ratio of the lipid components DSPC:cholesterol:PEG-DMG is 65:30:5.

21. The composition of claim 18, wherein the composition is a liquid.

22. The composition of claim 21, wherein the composition is a frozen liquid.

23. The composition of claim 21, wherein the composition is a lyophilized composition.

24. The composition of claim 18, wherein the active agent is present at 0.1-10 mg/mL of the final composition.

25. The composition of claim 18, wherein the composition is a frozen liquid and the active agent is present at 0.1-10 mg/mLof the final composition.

26. A pharmaceutical formulation comprising the composition of claim 18 and a cryoprotectant selected from sucrose, trehalose, raffinose, stachyose, verbascose, mannitol, glucose, lactose, maltose, maltotriose-heptaose, dextran, hydroxyethyl starch, insulin, sorbitol, glycerol, arginine, histidine, lysine, proline, dimethylsulfoxide and any combination thereof.

27. A method for preparing the composition of claim 18, comprising:
(a) providing one or more aqueous solutions in one or more reservoirs;
(b) providing one or more organic solutions in one or more reservoirs, wherein one or more of the organic solutions comprise a lipid;
(c) mixing the one or more aqueous solutions with the one or more organic solutions in a first mixing region, wherein the first mixing region is a confined volume mixing region; and
wherein the one or more aqueous solutions and the one or more organic solutions are introduced tangentially into a mixing chamber within the confined volume mixing region to produce a lipid nanoparticle solution; and
(d) dissolving an active agent in the lipid nanoparticle solution to form a lipid nanoparticle composition encapsulating the active agent.

28. The method of claim 27, wherein the confined volume mixing region of step (c) is a Multi-Inlet Vortex Mixer (MIVM).

29. The method of claim 27, wherein one or more aqueous solutions of step (a) comprises 50 to 300 mM ammonium sulfate.

30. The method of claim 27, wherein the lipid nanoparticle solution of step (c) is purified to remove free ammonium sulfate before being used in step (d).

31. The method of claim 27, wherein the active agent is at a concentration of 0.1 to 10 mg/mL of the final composition.

32. The method of claim 27, wherein the solution of step (d) containing the active agent is heated at 55-65° C. for 1-5 hours.

33. The method of claim 32, wherein the heated solution is cooled to about 22° C. after heating.

34. The method of claim 27, comprising:
(a) providing three aqueous solutions in one or more reservoirs, wherein one or more of the aqueous solutions comprise 50 to 300 mM ammonium sulfate;
(b) providing one organic solution in a reservoir, wherein the organic solution comprises DSPC, cholesterol and PEG-DMG;
(c) mixing the three aqueous solutions with the organic solution in a first mixing region, wherein the first mixing region is a confined volume mixing region; and
wherein the three aqueous solutions and the one organic solution are introduced tangentially into a mixing chamber within the confined volume mixing region to produce a lipid nanoparticle solution;
(d) purifying the lipid nanoparticle solution from the previous step to remove free ammonium sulfate; and
(e) dissolving an active agent in the purified lipid nanoparticle solution to form a lipid nanoparticle composition encapsulating the active agent.

35. The method of claim 34, wherein the confined volume mixing region of step (c) is a Multi-Inlet Vortex Mixer (MIVM).

36. A method for treating a human or animal disease comprising administering the lipid composition of claim 18 to said human or animal.

37. The method of claim 18, where the human or animal disease is cancer.
